(19) United States
(12) Patent Application Publication
(10) Pub. No.: US 2012/0330140 A1
(43) Pub. Date: Dec. 27, 2012

(54) TOMOGRAM OBSERVATION APPARATUS, PROCESSING METHOD, AND NON-TRANSITORY COMPUTER-READABLE STORAGE MEDIUM

(75) Inventor: Keiko Yonezawa, Kawasaki-shi (JP)

(73) Assignee: CANON KABUSHIKI KAISHA, Tokyo (JP)

(21) Appl. No.: 13/578,922

(22) PCT Filed: Mar. 9, 2011
(86) PCT No.: PCT/JP2011/056136

§ 371 Date: Aug. 14, 2012

(30) Foreign Application Priority Data
Apr. 22, 2010 (JP) 2010-099066

(51) Int. Cl. A61B 6/02

(52) U.S. Cl. 600/425

(57) ABSTRACT

A tomogram observation apparatus characterized by comprising: detection means for detecting a region in which an optic nerve extends from a retina layer of an eye to be examined to outside the eye to be examined; and generation means for generating a two-dimensional tomogram of a portion around an optic papilla of the eye to be examined, based on a position of the region.

### TECHNICAL FIELD

The present invention relates to a tomogram observation apparatus, a processing method, and a non-transitory computer-readable storage medium.

### BACKGROUND ART

Recently, departments of ophthalmology have adopted an apparatus called an optical coherence tomography (OCT), which acquires retinal tomograms. Obtaining retinal tomograms allows to quantitatively acquire a change in each layer along with the progress of a disease. This technique is therefore expected to enable comprehension of the degree of progress of a disease with higher accuracy and the evaluation of the effect of a medical treatment.

In glaucoma diagnosis, it is important to grasp a slight change in retina layer thickness. Such changes have been conventionally grasped by indices concerning a portion around the optic papilla which are called a C/D ratio (Cup/Disc ratio) and R/D ratio (Rim/Disc ratio) (Japanese Patent Laid-Open No. 2008-154951).

In contrast to this, an OCT uses a method called circle scan which acquires a tomogram along a concentric circle about several mm away from the center of the papilla. This method allows evaluation of a nerve fiber layer thickness which is thought to more accurately indicate a change in the development of glaucoma.

There is also known a method of reconstructing a tomogram along the position of a circle scan from OCT volume data instead of scanning on a circle. There is another known method which aligns a tomogram with a fundus image and reconstructs a tomogram at an arbitrary position designated on the fundus image, thereby presenting the tomogram (Japanese Patent Laid-Open No. 2008-73099).

In a follow-up, to evaluate a change in layer thickness, it is desired to accurately evaluate tomograms at the same position. A three-dimensional (3D) tomogram contains a lot of information. On the other hand, even a slight difference between cut two-dimensional slices will lead to tomograms exhibiting different aspects. For this reason, a change over time may be confused with a change due to the shift between cut slices. This poses an obstacle with respect to a quantitative follow-up.

In the above circle scan, it is difficult to scan the same position as that on a past image. Assume that a tomogram is to be reconstructed at the position of a circle scan. In this case, if a shift has occurred in the detection of the center of the papilla or the imaging directions at the time of imaging by the OCT differ from each other, it is difficult to acquire tomograms corresponding to the same portion of the retina.

### SUMMARY OF INVENTION

The present invention provides a technique of generating a tomogram by using a specific portion (an anatomical structure exhibiting small changes along with the progress of a disease) within a tomogram showing the three-dimensional shape of the retina.

According to a first aspect of the present invention there is provided a tomogram observation apparatus characterized by comprising: detection means for detecting a region in which an optic nerve extends from a retina layer of an eye to be examined to outside the eye to be examined; and generation means for generating a two-dimensional tomogram of a portion around an optic papilla of the eye to be examined, based on a position of the region.

According to a second aspect of the present invention there is provided a tomogram observation apparatus characterized by comprising: generation means for generating a first two-dimensional tomogram and a second two-dimensional tomogram of a portion around a retina layer of an eye to be examined; alignment means for aligning the first two-dimensional tomogram with the second two-dimensional tomogram by associating at least a partial region of a layer structure of a retina between a retinal pigment epithelium and a scleral layer of the eye to be examined; and display control means for causing display means to display the first two-dimensional tomogram and the second two-dimensional tomogram which are aligned with each other.

According to a third aspect of the present invention there is provided a processing method for a tomogram observation apparatus, characterized by comprising: the step of detecting a region in which an optic nerve extends from a retina layer of an eye to be examined to outside the eye to be examined; and the step of generating a two-dimensional tomogram of a portion around an optic papilla of the eye to be examined, based on the region.

According to a fourth aspect of the present invention there is provided a non-transitory computer-readable storage medium storing a computer program for causing a computer incorporated in a tomogram observation apparatus to function as detection means for detecting a region in which an optic nerve extends from a retina layer of an eye to be examined to outside the eye to be examined, and generation means for generating a two-dimensional tomogram of a portion around an optic papilla of the eye to be examined, based on the region.

Further features of the present invention will be apparent from the following description of exemplary embodiments (with reference to the attached drawings).

### BRIEF DESCRIPTION OF DRAWINGS

The accompanying drawings, which are incorporated in and constitute a part of the specification, illustrate embodiments of the invention, and together with the description, serve to explain the principles of the invention.

FIG. 1 is a view showing an example of the overall arrangement of a diagnosis support system according to an embodiment of the present invention;

FIG. 2 is a block diagram showing an example of the functional arrangement of a diagnosis support apparatus 10 shown in FIG. 1;

FIGS. 3A and 3B are views showing an outline of the manner in which a detection unit 14a in FIG. 2 detects specific portions;

FIG. 4 is a flowchart showing an example of a processing procedure in the diagnosis support apparatus 10 shown in FIG. 1;

FIG. 5 is a flowchart showing an example of a processing procedure in step S104 in FIG. 4;

FIGS. 6A to 6D are views showing an example of an outline of association processing;

FIG. 7 is a view showing an example of an outline of association processing; and

FIGS. 8A to 8C are views each showing an example of a display form.

### DESCRIPTION OF EMBODIMENTS

Exemplary embodiments of the present invention will now be described in detail with reference to the drawings. It should be noted that the relative arrangement of the components, the numerical expressions and numerical values set forth in these embodiments do not limit the scope of the present invention unless it is specifically stated otherwise.

### First Embodiment

FIG. 1 is a view showing an example of the overall arrangement of a diagnosis support system according to an embodiment of the present invention. Note that this embodiment will exemplify diagnosis support for follow-up of glaucoma.

In this diagnosis support system, a diagnosis support apparatus 10, a tomogram acquisition apparatus 20, and a data server 30 are connected to each other via a network 40 formed by a LAN (Local Area Network) or the like. Note that the respective apparatuses need not always be connected to each other via the network 40 as long as they can communicate with each other. For example, they can be connected to each other via a USB (Universal Serial Bus), IEEE1394, or the like, or may be connected to each other via a WAN (Wide Area Network).

In this case, the tomogram acquisition apparatus 20 is implemented by a time-domain OCT or Fourier-domain OCT, and has a function of obtaining a tomogram showing the three-dimensional shape of the retina. The tomogram acquisition apparatus 20 images the eye to be examined in diagnosis in accordance with operation by an operator (doctor or technician). The apparatus then transmits the image obtained by imaging to the diagnosis support apparatus 10 or the data server 30.

The data server 30 has a function of storing various kinds of data. The data server 30 stores, for example, three-dimensional (3D) tomograms obtained by obtaining tomograms of the macular region and optic papilla by the OCT, measurement results on visual field sensitivity by a perimeter, and the values of intraocular pressures, angles, visual acuities, and axial lengths of the eyes to be examined.

The diagnosis support apparatus 10 functions as a tomogram observation apparatus, which is used by an operator (doctor) for diagnosis in follow-up of glaucoma. The diagnosis support apparatus 10 associates three-dimensional tomograms obtained at different times by using the anatomical structure of a specific portion (the shape of the optic papilla rim or the structure of the retinal pigment epithelium) which is scarcely influenced by the progress of glaucoma. The apparatus then displays (presents) a two-dimensional (2D) tomogram of the layer structure including the nerve fiber layer around the optic papilla to the operator. A two-dimensional tomogram is an important index for the evaluation of the degree of the progress of glaucoma. This allows the doctor to easily evaluate the progress of glaucoma and perform an accurate follow-up.

Note that the diagnosis support apparatus 10, tomogram acquisition apparatus 20, and data server 30 described above incorporate computers. Each computer includes a main control unit such as a CPU and storage units such as a ROM (Read Only Memory), a RAM (Random Access Memory), and an HDD (Hard Disk Drive). The computer also includes input/output units such as a keyboard, mouse, display, buttons, and touch panel. These constituent units are connected to each other via a bus. The main control unit controls them by executing programs stored in the storage unit.

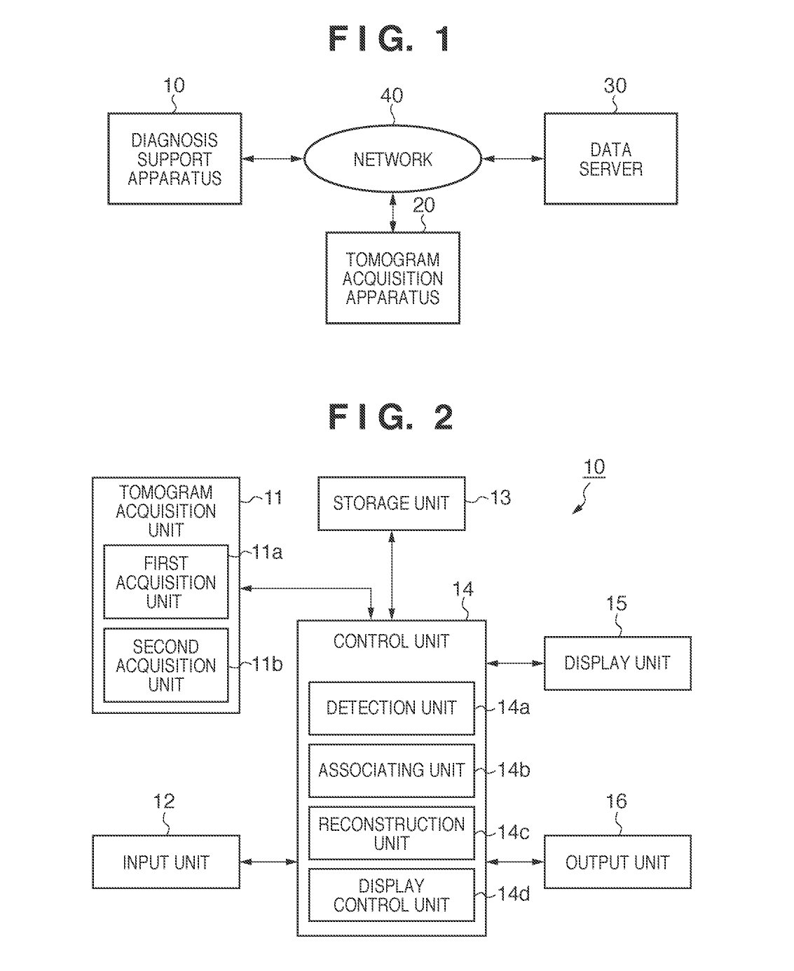

An example of the functional arrangement of the diagnosis support apparatus 10 shown in FIG. 1 will be described next with reference to FIG. 2.

The functional arrangement of the diagnosis support apparatus 10 includes a tomogram acquisition unit 11, an input unit 12, a storage unit 13, a control unit 14, a display unit 15, and an output unit 16.

The tomogram acquisition unit 11 has a function of acquiring a tomogram of the eye to be examined and includes a first acquisition unit 11a and a second acquisition unit 11b. The first acquisition unit 11a acquires a tomogram (to be referred to as the first tomogram hereinafter) of the eye to be examined as a diagnosis target. The second acquisition unit 11b acquires a tomogram (to be referred to as the second tomogram hereinafter) of the eye to be examined as a comparative target of the first tomogram. The first acquisition unit 11a and the second acquisition unit 11b acquire tomograms from the tomogram acquisition apparatus 20 or the data server 30 based on information (for example, the name, age, and sex of the patient) relating to the eye to be examined input by the operator. Assume that in this case, tomograms acquired by the first acquisition unit 11a and the second acquisition unit 11b are tomograms of the same eye to be examined, which are captured at different times. That is, the first and second tomograms are acquired for follow-up of the same eye to be examined.

The input unit 12 inputs an instruction from the operator (doctor or technician) to the apparatus. The storage unit 13 stores various kinds of information. The storage unit 13 also stores, for example, two-dimensional tomograms and the like in addition to information about the eye to be examined, three-dimensional tomograms, and information obtained from the input unit 12.

The display unit 15 is, for example, a display device such as a monitor, and displays various kinds of information to the doctor or the like. Note that the display unit 15 may be provided outside the diagnosis support apparatus 10. The output unit 16 outputs various kinds of information to the data server 30 and the like. The control unit 14 comprehensively controls the diagnosis support apparatus 10. In this case, the control unit 14 includes a detection unit 14a, an associating unit 14b, a reconstruction unit 14c, and a display control unit 14d.

The detection unit 14a detects a specific portion from each of the tomograms acquired by the first acquisition unit 11a and the second acquisition unit 11b. In this case, specific portions are portions used to associate the first tomogram with the second tomogram. Assume that in this embodiment, a specific portion is the rim portion of the optic papilla region (to be also referred to as the papilla rim hereinafter). Although described in detail later, the detection unit 14a detects the boundary of the retinal pigment epithelium, and detects the papilla rim based on the detected boundary. Note that the specific portion is not specifically limited to such a portion as long as it is a portion which is scarcely influenced by the progress of glaucoma.

In general, although the thickness of the retinal nerve fiber layer diminishes along the progress of glaucoma, the shape of the optic papilla rim portion or the structure of the retinal pigment epithelium (RPE) is relatively stable. For this reason, this embodiment associates three-dimensional tomograms with each other by using these structures which are scarcely influenced by the progress of a disease. This makes it possible to reconstruct a two-dimensional tomogram upon matching the positions and angles of three-dimensional tomograms obtained by imaging the same eye to be examined at different times.

In this case, the papilla rim is defined as the inside of the white scleral ring (Elschnig's scleral ring (scleral layer)) around the papilla which is ophthalmoscopically observed according to the glaucomatous optic disk retinal nerve fiber layer change determination guideline. As a method of detecting the papilla rim from the three-dimensional tomogram obtained by the OCT, a technique of detecting an end point of retinal pigment epithelium is known. This technique uses the fact that the end edge of the retinal pigment epithelium almost overlaps the papilla rim, and is regarded as an effective technique for detecting the papilla rim except for a case in which a parapapillaryatrophy (PPA) is observed.

Note that this embodiment exemplifies a case in which an end point of the retinal pigment epithelium is detected, and the papilla rim is detected based on the detection result. However, the method to be used is not limited to this, and it is possible to use another method of detecting the papilla rim. For example, it is possible to use a method of detecting the opening of a papilla portion (BMO: Bruch's Membrane Opening) by detecting the Bruch's membrane.

The associating unit 14b associates (aligns) three-dimensional tomograms by using the specific portions (the papilla rims) detected by the detection unit 14a. More specifically, the associating unit 14b associates the respective portions of the papilla rims with each other to associate the first and second tomograms with each other.

Note that tomograms are associated by using specific portions because of the possibility that features which severely change along the progress of glaucoma may have greatly changed since the first and second tomograms were obtained at different times. Even if tomograms are associated with each other by using the overall images, the obtained result may not be suited to the comparison of the two images. For this reason, this embodiment associates tomograms with each other by using features which exhibit small changes along with the progress of glaucoma, thereby allowing for comparison of features exhibiting large changes.

The reconstruction unit 14c generates (reconstructs) two-dimensional tomograms at predetermined positions (positions suited to comparison) on the respective three-dimensional tomograms which are associated with each other. That is, the reconstruction unit 14c cuts two-dimensional slices at corresponding positions on two-dimensional tomograms along a predetermined direction. With this operation, the reconstruction unit 14c generates two-dimensional tomograms.

The display control unit 14d generates each kind of frame and causes the display unit 15 to display it. The display control unit 14d causes the display unit 15 to display, for example, two-dimensional tomograms. An example of the functional arrangement of the diagnosis support apparatus 10 has been described above.

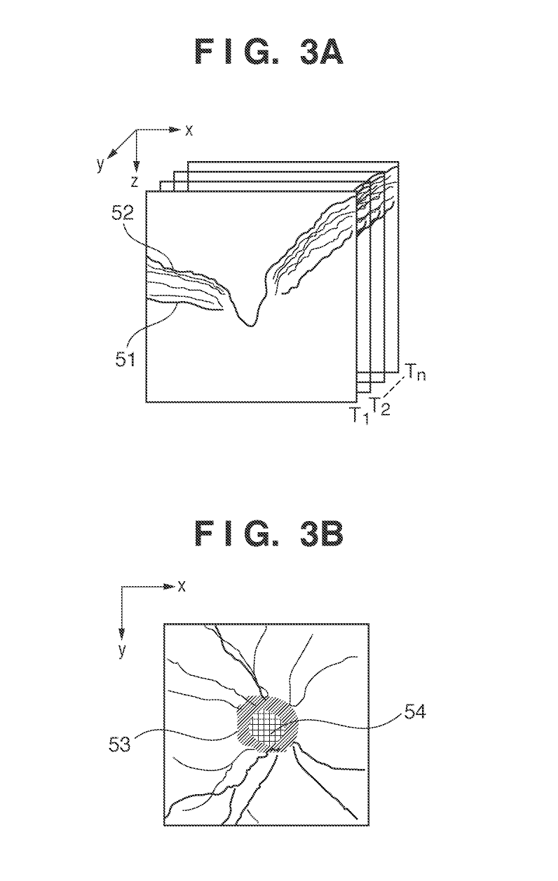

An outline of the manner in which the detection unit 14a shown in FIG. 2 detects specific portions will be described next with reference to FIGS. 3A and 3B. FIGS. 3A and 3B respectively show examples of a tomogram and projected image of the optic papilla captured by the OCT.

FIG. 3A shows tomograms of the optic papilla captured by the OCT. Reference symbols T1 to Tn denote two-dimensional tomograms (B-scan images) of the optic papilla. Reference numeral 52 denotes the inner limiting membrane; and 51, the boundary of the retinal pigment epithelium. FIG. 3B shows the projected image generated by integrating the luminance values of tomograms in the depth direction (z direction). Reference numeral 53 denotes the optic papilla rim (Disc); and 54, the rim of the cavity (Cup).

When detecting specific portions, the detection unit 14a aligns the tomograms (tomograms T1 to Tn) shown in FIG. 3A. The detection unit 14a performs this alignment by using an evaluation function for obtaining the similarity between adjacent tomograms. The detection unit 14a changes the relative positions of images so as to make the value calculated by using this evaluation function satisfy a predetermined condition.

The detection unit 14a then detects the boundary 51 of the retinal pigment epithelium from an aligned three-dimensional tomogram. The boundary 51 of the retinal pigment epithelium is a high-luminance region, and hence may be detected by using a Hessian filter or an edge detection filter.

In this manner, the detection unit 14a detects the boundary 51 of the retinal pigment epithelium, and detects an end of the retinal pigment epithelium near the optic papilla from the boundary 51 of the retinal pigment epithelium. The detected end of the retinal pigment epithelium is then coupled in the three-dimensional region, thereby obtaining an optic papilla rim (Disc) 53. The detection unit 14a stores the detection result in the storage unit 13. With this operation, the apparatus terminates the detection processing by the detection unit 14a.

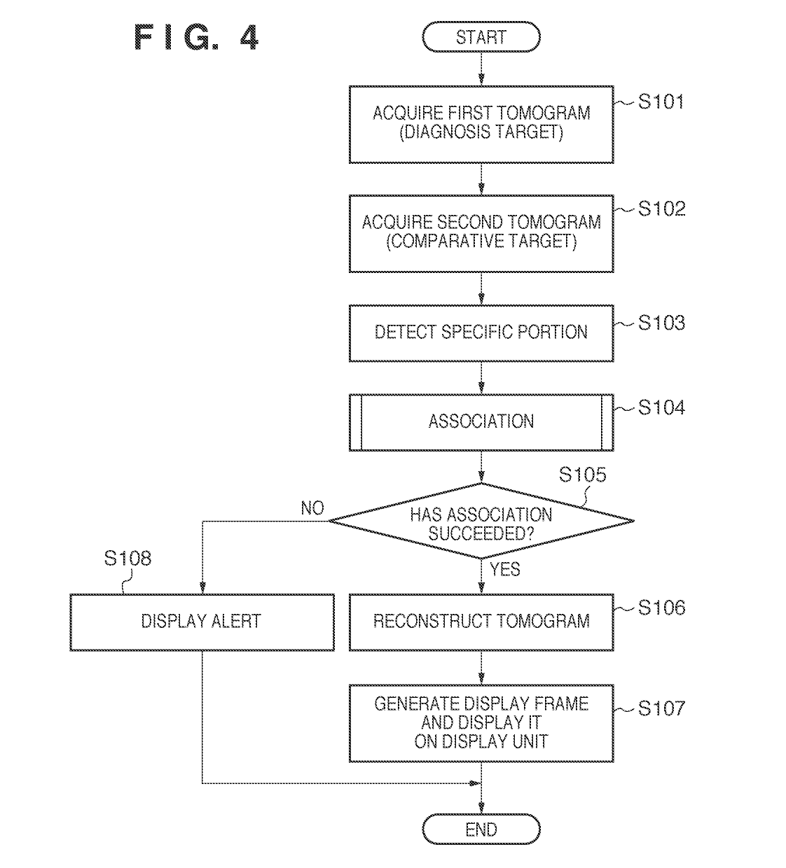

An example of a processing procedure in the diagnosis support apparatus 10 shown in FIG. 1 will be described next with reference to FIG. 4.

The diagnosis support apparatus 10 causes the first acquisition unit 11a to acquire a three-dimensional tomogram (first tomogram) of the eye to be examined as a diagnosis target from the tomogram acquisition apparatus 20 or the data server 30 (S101). The diagnosis support apparatus 10 also causes the second acquisition unit 11b to acquire a three-dimensional tomogram (second tomogram) of the eye to be examined as a comparative target from the data server 30 (S102). Note that this apparatus acquires a tomogram of the eye to be examined based on identification information (for example, an object identification number) for identifying the eye to be examined.

Subsequently, the diagnosis support apparatus 10 causes the detection unit 14a to detect specific portions from the first and second tomograms (S103). That is, the detection unit 14a detects the boundary of the retinal pigment epithelium, and detects the papilla rim based on the detection result. The diagnosis support apparatus 10 causes the associating unit 14b to associate the first and second tomograms by using the detected papilla rims (S104).

The association processing in step S104 will be described below with reference to FIG. 5.

When starting the association processing, the associating unit 14b masks regions of several pixels to several tens of pixels on the inside and outside of the papilla boundary as regions where the boundary of the retinal pigment epithelium does not exist. The associating unit 14b then performs paraboloid approximation of the boundary of the retinal pigment epithelium by using the remaining regions (S201). In this case, the size of a region to be masked on an outside portion of the papilla boundary depends on the size of the papilla, and is set to, for example, 1/10 the longitudinal diameter of the papilla.

In paraboloid approximation in a three-dimensional space, the parameters to be used include the coordinates (x₀, y₀, z₀) of the origin, rotation (Θ, φ, ψ), and (k₁, k₂) indicating the curvature of a paraboloid. As described above, since the first and second tomograms are captured at different times, the coordinates of the origins and the rotations are likely to differ from each other due to the influences of the differences between imaging parameters, the movement of the eye, and the like. In contrast to this, the curvatures remain almost the same values on the first and second tomograms because the structure of the eyeball does not greatly change along the progress of glaucoma.

First of all, therefore, the associating unit 14b performs paraboloid approximation of the first tomogram (a tomogram of the eye to be examined as a diagnosis target), and then obtains the coordinates of the origin and rotation of the second tomogram (a tomogram of the eye to be examined as a comparative target) by approximation. Assume that the curvature is the same value as that obtained from the first tomogram. Note that the sequence of processing is not specifically limited. For example, it is possible to obtain a curvature from the second tomogram (comparative eye) and then obtain only the coordinates of the origin and rotation from the first tomogram (target eye). In addition, the manner of calculating an approximate curved surface is not limited to this technique. For example, it is possible to approximate a more complicated shape by using the thin-plate spline.

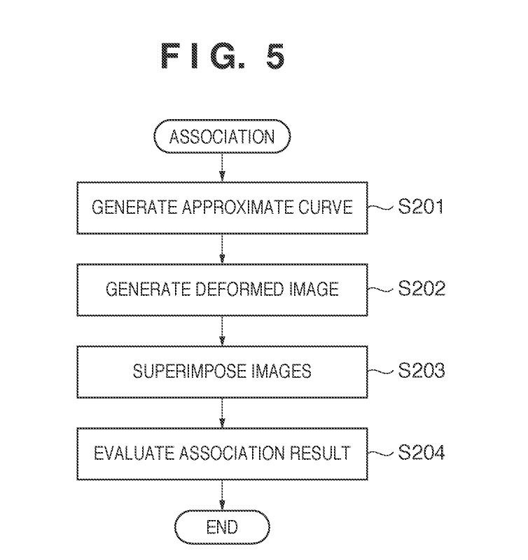

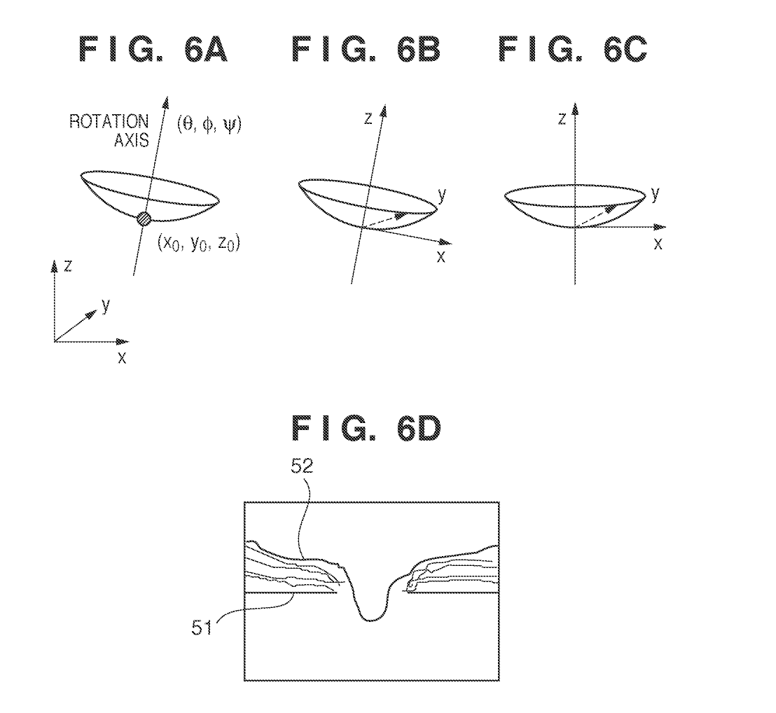

The associating unit 14b then deforms a three-dimensional tomogram so as to make the boundary of the retinal pigment epithelium horizontal, based on the paraboloid obtained in step S201 (S202). It is possible to perform this deformation by transforming the vertex of the paraboloid (the boundary of the approximate retinal pigment epithelium obtained in step S201) into an origin by affine transformation and matching the rotation axis with the z-axis. For the sake of descriptive convenience, this embodiment will be described on the assumption that the magnification and resolution of the first tomogram are the same as those of the second tomogram at the time of imaging. However, the settings of the two tomograms at the time of imaging may differ from each other. In this case, it is possible to perform coordinate transformation in consideration of the differences between the magnifications and resolutions of the two tomograms. With regard to the rotation ψ around the rotation axis, the imaging direction of a tomogram is set as the x-axis (see FIGS. 6A to 6C).

The associating unit 14b then moves the paraboloid in the z direction so as to set the position of the paraboloid on an x-y plane. This deforms the approximate paraboloid of the boundary 51 of the retinal pigment epithelium to make it horizontal (see FIG. 6D).

More specifically, the approximate paraboloid in the state shown in FIG. 6C can be expressed by

z=f(x,y)=ax²+bxy+cy² (1)

The value of each pixel is then changed as indicated by

I(x,y,z)=I_{ORG}(x,y,z+f(x,y)) (2)

This makes each pixel on the approximate paraboloid move on the x-y plane. As a result, all the pixel values move in the z direction. This makes it possible to obtain the tomogram shown in FIG. 6D.

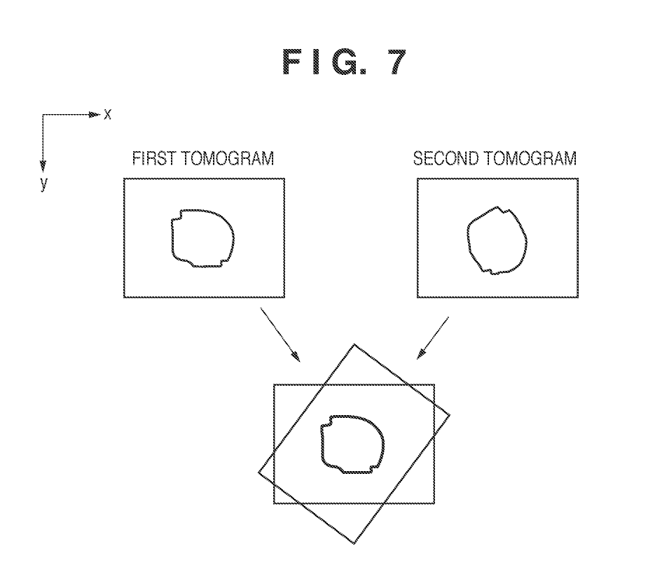

Subsequently, the associating unit 14b superimposes the first tomogram (target eye) and the second tomogram (comparative eye) on each other based on the deformed image obtained in step S202 (S203). More specifically, the associating unit 14b projects the papilla rim on the x-y plane and transforms the papilla rim detected in the three-dimensional space into a shape on a two-dimensional plane. As shown in FIG. 7, the associating unit 14b rotates the second tomogram (comparative eye) about the origin (x₀, y₀, z₀) within the x-y plane. With this operation, the associating unit 14b superimposes the tomograms such that the shapes of the papilla rims of the respective projected images projected on the x-y plane satisfy a predetermined condition (almost coincide with each other).

In this superimposition processing, control points are set, at predetermined intervals, on the papilla rims of the first and second tomograms projected on the x-y plane. The associating unit 14b obtains the sum total (square sum) of the distances between control points corresponding to the first and second tomograms. In this case, the associating unit 14b obtains a rotational angle that minimizes the square sum of the distances between the corresponding control points while rotating the second tomogram (comparative eye) on the x-y plane relative to the first tomogram (target eye). In this manner, the associating unit 14b associates the papilla rim of the first tomogram (target eye) with the papilla rim of the second tomogram (comparative eye).

Note that control points are set based on the shapes of detected papilla rim portions. More specifically, two points with the largest distance between them are selected on a closed surface as a papilla rim portion, one of the two points which is located higher than the other (an upper portion of the face) is set as a start point (C1), and N points (C1 to CN) are set at predetermined intervals. In this case, if it is thought that there is no large change in the shape of the papilla rim portion between the first tomogram and the second tomogram, control points whose numbers coincide with each other are regarded as corresponding control points. There is available another method in which when a papilla rim portion has a characteristic shape, a corresponding point is detected and set as a start point (C1). In this case as well, corresponding control points are those having numbers coinciding with each other.

Upon completing the superimposition processing, the associating unit 14b evaluates the association result (S204). More specifically, if the square sum (its minimum value) of the distances between control points set on the papilla rim in the processing in step S203 exceeds a predetermined value (threshold), the associating unit 14b determines that the association processing has failed. If the square sum of the distances between the control points falls within the range of the threshold, the associating unit 14b determines that the association processing has succeeded. In this case, the threshold changes depending on the resolution of images or the like.

Since the value obtained by dividing the square sum of the distances between control points by the number of control points is preferably equal to or more than about 10, a threshold may be set based on this. That is, if the average of the distances between corresponding control points on images having a resolution of about 10 μm per pixel is equal to or more than 10-odd pixels, the associating unit 14b determines that the association processing has failed.

In this manner, the associating unit 14b associates the papilla rims detected from the first tomogram (target eye) and the second tomogram (comparative eye) with each other and evaluates the association result. The associating unit 14b then terminates this association processing (the processing shown in FIG. 5). Note that the storage unit 13 stores, as the association result, information such as parameters representing the approximate paraboloids of retinal nerve fiber layer boundaries, the papilla rims associated with each other, and the correspondence relationship between the control points set on the papilla rims on the respective tomograms.

Referring back to FIG. 4, when completing the association processing, the diagnosis support apparatus 10 causes the associating unit 14b to determine whether the above association processing has succeeded. That is, the associating unit 14b determines whether a value indicating the association result (the square sum of the distances between corresponding control points) falls within the range of the predetermined value (threshold).

Upon determining that the association processing has failed (NO in step S105), the diagnosis support apparatus 10 causes the display control unit 14d to display the corresponding information on the display unit 15 (S108). If the association processing has failed, it is highly possible that the shape of the papilla rim has greatly changed between the first tomogram and the second tomogram. For this reason, the apparatus may display an alert concerning the progress of a retinal disease. For example, the apparatus displays an alert suggesting the possibility of a concurrent disease other than glaucoma.

Upon determining that the association processing has succeeded (YES in step S105), the diagnosis support apparatus 10 causes the reconstruction unit 14c to determine the direction in which each tomogram is to be reconstructed, and generates a two-dimensional tomogram along the direction. More specifically, the reconstruction unit 14c generates (reconstructs) each two-dimensional tomogram in the direction from the papilla rim in the three-dimensional tomogram to the rotation axis of the approximate paraboloid obtained in step S201 in FIG. 5. With this operation, the apparatus generates a two-dimensional tomogram based on the first tomogram and a two-dimensional tomogram based on the second tomogram. Note that when generating a tomogram, the apparatus may perform image interpolation based on, for example, the bicubic method for an image positioned at coordinates which have not been acquired at the time of imaging.

Upon completing the reconstruction of two-dimensional tomograms, the diagnosis support apparatus 10 causes the display control unit 14d to generate a display image based on the reconstructed two-dimensional tomograms (S106). In this case, the operator (doctor) needs to generate a display image so as to easily grasp a feature which greatly changes along the progress of glaucoma.

Subsequently, the diagnosis support apparatus 10 causes the display control unit 14d to display, on the display unit 15, a display frame having two-dimensional tomograms arranged side by side based on the generated display image (S107). Note that this reconstruction result and the like are stored in the storage unit 13, or are stored in the data server 30 by the output unit 16.

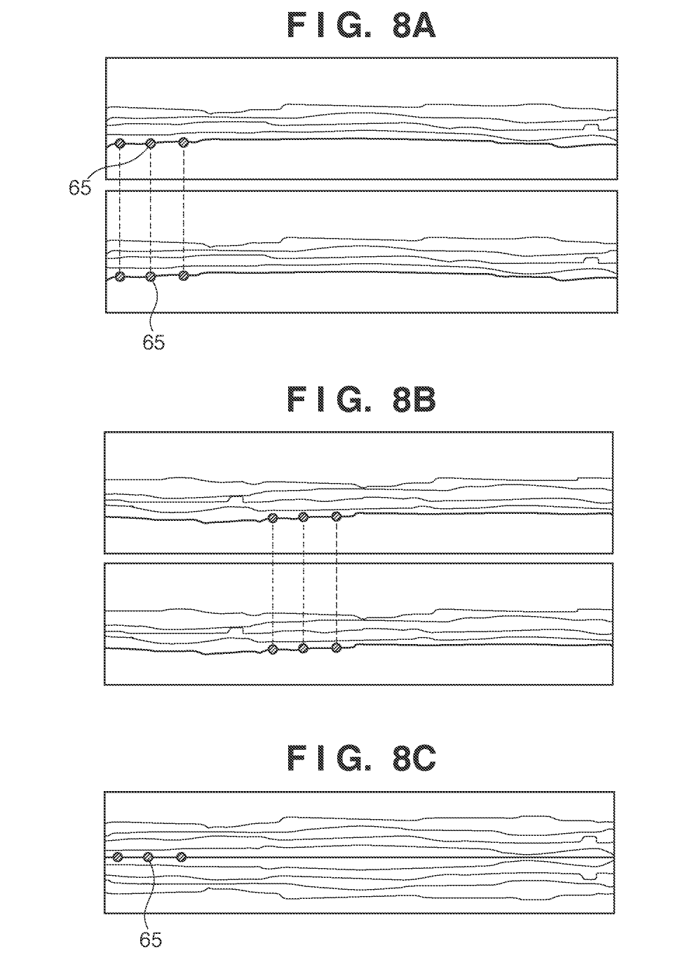

An example of a frame to be displayed in step S107 in FIG. 4 will be described next with reference to FIGS. 8A to 8C.

As shown in FIG. 8A, the display unit 15 displays, as an example of a display frame, the two-dimensional tomogram reconstructed based on the first tomogram and the two-dimensional tomogram reconstructed based on the second tomogram side by side. At this time, the respective two-dimensional tomograms are associated with each other by association processing (step S104 in FIG. 4) using control points 65 set on the papilla rims of the respective three-dimensional tomograms. That is, the respective portions of the papilla rims on the two-dimensional tomograms are associated with each other. Matching the positions of the corresponding control points in the lateral direction in FIG. 8A (papilla rims) with each other can make a feature which has changed between the tomograms conspicuous.

It is known that the sizes of papillae greatly vary among individuals. Findings to be noted in diagnosis vary depending on the difference in size between the papillae. For this reason, for example, the width of the display area of each two-dimensional tomogram may be changed in accordance with the circumferential length of the Papilla rim. This makes it possible to allow the operator (doctor) to intuitively know the difference in size between the papilla rims in diagnosis.

In addition, each papilla rim is formed by a continuous closed curve close to a circle surrounding the papilla. FIG. 8A is a sectional view taken along the circumference of the closed curve. For this reason, the apparatus may be configured to allow the operator to designate a specific position as a start point (the left end in FIG. 8A) on the closed curve by operator designation (mouse operation). When, for example, the operator scrolls to the right while clicking the mouse, the apparatus may slide the display of a two-dimensional tomogram to the right, as shown in FIG. 8B.

In order to grasp the progress of glaucoma, it is important to grasp a change in retina layer structure. To explicitly present this change to the operator, this apparatus may display only retina information by eliminating an image below the retinal pigment epithelium (on the choroid membrane side).

For this purpose, as shown in FIG. 8C, the apparatus may display only the upper portions of the retinal pigment epitheliums so as to make them face each other. In this case, after the boundaries of the retinal pigment epitheliums shown in FIG. 8A are linearized, one of the images is flipped vertically. This can present a change in retina layer more clearly to the operator (doctor).

As described above, the first embodiment associates the first and second tomograms by using a specific portion in each tomogram (an anatomical structure exhibiting small changes along with the progress of a disease). The apparatus then reconstructs two-dimensional tomograms along a predetermined direction at the same position (corresponding positions) in the two associated tomograms, and displays the reconstructed tomograms to the operator. This allows the doctor (operator) to accurately grasp the degree of the diminution of the retinal nerve fiber layer around the papilla.

Note that in the association processing in step S203 in FIG. 5 described above, the apparatus projects the papilla rims of the first and second tomograms on a two-dimensional plane, and associates the tomograms with each other based on control points set on the papilla rims on the projected images. However, the apparatus may use a method other than this.

For example, obtaining the integrated value of each pixel from a deformed image in the z-axis direction can also generate a projected image on an x-y plane. In this case, the apparatus obtains relative positions at which the projected images of the first and second tomograms are superimposed on each other with (highest) high similarity, and associates the papilla rims of the two tomograms with each other. Note that in this method, it is necessary to unify the numbers of pixels whose values are to be integrated. For this reason, if pixels fall outside the imaging area at the time of the generation of a deformed image, it is possible to obtain the integrated values of pixels in a rectangular parallelepiped including an x-y plane constituted by only effective pixels. It is possible to calculate similarities by using a generally used method. For example, there is available a method of binarizing images and superimposing them so as to maximize the number of pixels whose values coincide with each other. In this case, features such as blood vessels located above the retinal pigment epitheliums (on the inner limiting membrane side) are made to coincide with each other. Note, however, that if the direction of integration of projected images greatly differs from the z-axis of a three-dimensional image, the shadows of the blood vessels formed at the time of imaging may affect other portions. This causes an error at the time of superimposition. It is, however, possible to reduce the influences of the shadows of the blood vessels by extracting blood vessel regions in advance and evaluating similarity upon masking regions in the projected images which are affected by the shadows of the blood vessels.

Owing to the diminution of the retina layer along with the progress of glaucoma, it is expected that feature amounts above the retinal pigment epitheliums may greatly change relative to images captured in the past. This tendency is especially noticeable near the optic papilla. In order to suppress the influences of such changes on association processing, it is possible to obtain relative positions at which the first and second tomograms are superimposed on each other with the highest similarity by using feature amounts existing only near and below the retinal pigment epitheliums. In this case, it is possible to evaluate similarity on a two-dimensional plane by generating projected images in the above manner or by using three-dimensional volumes without any change. In addition, to remove a region exhibiting a large change, it is possible to mask a region located inward from the outside of the papilla by a distance of several pixels to several tens pixels and also mask a region in a projected image which is affected by a blood vessel. This makes it possible to perform accurate superimposition.

Furthermore, association processing using a technique other than those described above includes, for example, a method of emphatically associating the optic papilla boundaries and a method of approximately obtaining a projecting plane from detection points on the papilla boundary. Note that the technique according to this embodiment described above is a means more effective than these techniques even if the positions and directions of the optic papillae in the first and second tomograms greatly differ from each other.

### Second Embodiment

The second embodiment will be described next. The first embodiment has exemplified the case in which images of the same eye to be examined which are captured at different times are compared in a follow-up. In contrast, the second embodiment will exemplify a case in which the left and right eyes of the same object are compared with each other. This is because the left and right eyes of the same object exhibit small variations in the sizes of the optic papillae. It is known that the sizes of the optic papillae greatly vary among individuals. In contrast, the left and right eyes of the same person exhibit small variations in the sizes of the optic papillae (it is reported that the differences in size between the left and right papillae of 99% people fall within 1 mm to 2 mm).

In this case, the second embodiment associates tomograms with each other with focus on the shapes of papilla boundaries. The second embodiment differs from the first embodiment in the association processing in step S104 in FIG. 4. Since the apparatus arrangement and processing other than the association processing are the same as those in the first embodiment, a description of them will be omitted.

The papilla rim has a shape approximated by an ellipse longer vertically than horizontally. When the apparatus detects the papilla rims from three-dimensional tomograms (first and second tomograms) as in step S103 in FIG. 4 in the first embodiment, the shape of each papilla rim is a closed curve in a three-dimensional space. Considering that the left and right papillae do not greatly vary in size, the papilla rims are detected from the two eyes as closed curves having similar shapes. In this case, many of the detected papilla rims differ in position and direction in a three-dimensional space. In this case it is thought, in consideration of the movement of the eyes and differences in imaging parameters, that many of the detected papilla rims differ in position and direction.

If the detected papilla rim can be approximated by a two-dimensional closed curve, a projected image is preferably formed on the corresponding plane. The apparatus then obtains an approximate plane of each papilla rim so as to minimize the sum total of the distances of detection points on each of the papilla rims of the left and right eyes from the approximate plane. The apparatus generates projected images of the three-dimensional tomograms on the approximate plane obtained in this manner, and aligns the projected papilla rims with each other. This makes it possible to perform association.

The apparatus may use a more simplified method, that is, selecting several detection points from the detected detection points on the papilla rims, and associating them with each other by using a straight line orthogonal to line segments connecting the detection points as a normal vector. More specifically, the apparatus obtains two detection points A and B whose distance between them is the largest and other two detection points C and D at positions almost orthogonal to a line segment connecting the two detection points A and B. The apparatus then obtains a vector orthogonal to both a vector AB and a vector DC as a normal vector. It is possible to obtain a projected image by performing projection along this vector.

In this case, it is possible to select an arbitrary direction as the direction in which two-dimensional tomograms are reconstructed. When, for example, alignment is performed on a two-dimensional plane, the projecting direction at the time of generation of two-dimensional tomograms from a three-dimensional tomogram may be set as a reconstruction direction. In addition, for example, when alignment is performed in a three-dimensional space, a direction orthogonal to an approximate closed surface corresponding to the papilla rim may be set as a reconstructing direction.

As described above, according to the second embodiment, the apparatus associates three-dimensional tomograms obtained by imaging the left and right eyes of the same person and reconstructs two-dimensional tomograms at corresponding positions on the two tomograms. This can clearly present the differences between the left and right eyes to the operator (doctor) when glaucoma has occurred only in one eye or the progress of glaucoma in the left eye differs from that in the right eye.

### Third Embodiment

The third embodiment will be described next. The third embodiment will exemplify a case in which tomograms to be compared are processed to clarify the differences between the tomograms and present them to the operator. More specifically, when displaying tomograms to be compared, the apparatus executes difference processing to display the differences between the two tomograms to the operator. This embodiment differs from the first and second embodiments in the display control processing shown in step S107 in FIG. 4. Since the apparatus arrangement and processing other than the display control processing are the same as those in the first and second embodiments, a description of them will be omitted.

In this case, it is possible to generate a difference image by, for example, subtracting, from the luminance values of the respective pixels of the first tomogram, the luminance values of the corresponding pixels of the second tomogram. In contrast to this, it is also possible to generate a difference image by, for example, subtracting, from the luminance values of the respective pixels of the second tomogram, the luminance values of the corresponding pixels of the first tomogram.

An example of the display form of a difference image will be described below. For example, it is possible to perform display in the form shown in FIG. 8A. For example, the apparatus displays a tomogram as a diagnosis target (a two-dimensional tomogram based on the first tomogram) in the upper area, and displays a difference image in the lower area. Note that the display form to be used is not limited to this. The method to be used is not specifically limited as long as it is possible to display a tomogram as a diagnosis target and a difference image on the same frame and to easily compare the two images.

As described above, the third embodiment obtains the differences between the first and second tomograms and displays the differences between the two tomograms to the operator. In this case as well, the same effects as those described above are obtained.

The typical embodiments of the present invention have been described above. However, the present invention is not limited to the embodiments described above and shown in the accompanying drawings, and can be modified and executed as needed within the spirit and scope of the invention.

For example, the above embodiments have exemplified the case in which the first tomogram (a tomogram of the eye to be examined as a diagnosis target) and the second tomogram (a tomogram of the eye to be examined as a comparative target) are associated with each other by using specific portions, and the two-dimensional tomograms are displayed. However, the present invention is not limited to this. For example, it is possible to detect a specific portion from any of the tomograms and generate a two-dimensional tomogram based on the specific portion. That is, it is not necessary to associate a plurality of tomograms (first and second tomograms). In this case as well, since a two-dimensional tomogram is generated based on a specific portion (its position), even if there are a plurality of tomograms captured at different times, two-dimensional tomograms at the same position are obtained.

### Other Embodiments

Aspects of the present invention can also be realized by a computer of a system or apparatus (or devices such as a CPU or MPU) that reads out and executes a program recorded on a memory device to perform the functions of the above-described embodiments, and by a method, the steps of which are performed by a computer of a system or apparatus by, for example, reading out and executing a program recorded on a memory device to perform the functions of the above-described embodiments. For this purpose, the program is provided to the computer for example via a network or from a recording medium of various types serving as the memory device (for example, computer-readable storage medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions. This application claims the benefit of Japanese Patent Application No. 2010-099066 filed on Apr. 22, 2010, which is hereby incorporated by reference herein in its entirety.

1-2. (canceled)

3. A tomogram observation apparatus comprising:
an acquisition unit configured to acquire a first three-dimensional tomogram and a second three-dimensional tomogram of a portion around a retina layer of an eye to be examined;
an alignment unit configured to align the first three-dimensional tomogram with the second three-dimensional tomogram by associating at least a partial region of a layer structure of a retina between a retinal pigment epithelium and a scleral layer of the eye to be examined;
a generating unit configured to generate a first two-dimensional tomogram and a second two-dimensional tomogram as two-dimensional tomograms at corresponding positions on the first three-dimensional tomogram and the second three-dimensional tomogram which are aligned with each other; and
a display control unit configured to cause a display unit to display the first two-dimensional tomogram and the second two-dimensional tomogram.

4-5. (canceled)

6. The apparatus according to claim 3, further comprising a detection unit configured to detect the boundary of the retinal pigment epithelium and detect at least the partial region of the layer structure of the retina based on the detected boundary.

7. The apparatus according to claim 3, said alignment unit comprising:
a setting unit configured to project at least the partial region of the layer structure of the retina detected from the first three-dimensional tomogram and the second three-dimensional tomogram on a two-dimensional plane, respectively, and to set control points, at predetermined intervals, on at least the partial region of the respective projected images projected on the two-dimensional plane;
a calculation unit configured to calculate the square sum of the distances between corresponding control points on a projected image based on the first three-dimensional tomogram and a projected image based on the second three-dimensional tomogram; and
a determination unit configured to determine that the association processing by said alignment unit has succeeded if a minimum value of the calculated square sum falls within the range of a predetermined threshold, and to determine that the association processing by said alignment unit has failed if the minimum value of the calculated square sum exceeds the predetermined threshold.

8. The apparatus according to claim 7, wherein if it is determined by the determination unit that the association processing by said alignment unit has failed, said display control unit causes said display unit to display an alert concerning the progress of the retinal disease.

9. The apparatus according to claim 3, wherein said display control unit executes a difference processing using the first two-dimensional tomogram and the second two-dimensional tomogram, and causes said display unit to display a difference image obtained by the difference processing.

10. A processing method for a tomogram observation apparatus, comprising:
the step of acquiring a first three-dimensional tomogram and a second three-dimensional tomogram of a portion around a retina layer of an eye to be examined;
the step of aligning the first three-dimensional tomogram with the second three-dimensional tomogram by associating at least a partial region of a layer structure of a retina between a retinal pigment epithelium and a scleral layer of the eye to be examined;
the step of generating a first two-dimensional tomogram and a second two-dimensional tomogram as two-dimensional tomograms at corresponding positions on the first three-dimensional tomogram and the second three-dimensional tomogram which are aligned with each other; and
the step of causing a display means unit to display the first two-dimensional tomogram and the second two-dimensional tomogram.

11. A non-transitory computer-readable storage medium storing a computer program for causing a computer incorporated in a tomogram observation apparatus to function as
an acquisition unit configured to acquire a first three-dimensional tomogram and a second three-dimensional tomogram of a portion around a retina layer of an eye to be examined;
an alignment unit configured to align the first three-dimensional tomogram with the second three-dimensional tomogram by associating at least a partial region of a layer structure of a retina between a retinal pigment epithelium and a scleral layer of the eye to be examined;
a generating unit configured to generate a first two-dimensional tomogram and a second two-dimensional tomogram as two-dimensional tomograms at corresponding positions on the first three-dimensional tomogram and the second three-dimensional tomogram which are aligned with each other; and
a display control unit configured to cause a display unit to display the first two-dimensional tomogram and the second two-dimensional tomogram.
